(19) United States
(12) Patent Application Publication
(10) Pub. No.: US 2013/0195911 A1
(43) Pub. Date: Aug. 1, 2013

(54) Compositions And Methods For Treating Skin

(71) Applicants: Christine M. Baier, Massapequa Park, NY (US); Chia-Wen Chen, Eastchester, NY (US); Lieve Declercq, Ekeren (BE); Susan M. Daly, Commack, NY (US); Elizabeth I. Starr, Great Neck, NY (US)

(72) Inventors: Christine M. Baier, Massapequa Park, NY (US); Chia-Wen Chen, Eastchester, NY (US); Lieve Declercq, Ekeren (BE); Susan M. Daly, Commack, NY (US); Elizabeth I. Starr, Great Neck, NY (US)

(21) Appl. No.: 13/579,980

(22) PCT Filed: Mar. 8, 2010
(86) PCT No.: PCT/US2010/026561

§ 371 Date: Feb. 6, 2013

(51) Int. Cl. A61K 8/97; A61K 36/06; A61Q 19/00; A61K 36/074; A61K 36/18; A61K 8/99; A61K 36/07

(52) U.S. Cl. CPC A61K 8/975; A61K 8/97; A61K 8/99; A61K 36/06; A61K 36/07; A61K 36/074; A61K 36/18; A61Q 19/00
USPC 424/195.15

(57) ABSTRACT

A skin treatment composition comprising a combination of at least one extract from the Hippophae genus and at least one extract from the Pleurotus genus and a method for treating sensitive skin for improvement comprising administering a topical composition comprising at least one extract from the Hippophae genus and at least one extract from the Pleurotus genus.

### TECHNICAL FIELD

The invention is in the field of compositions for application to skin which soothe skin and treat sensitive skin for improvement.

### BACKGROUND OF THE INVENTION

There are many environmental conditions that contribute to skin sensitivity, such as poor diet, stress, fatigue, pollution in the form of particulate matter in the air (e.g. auto exhaust, cigarette smoke), sun exposure, and so on. Response to such aggressors can result in sensitive skin, which includes conditions such as blemishes, uneven pigmentation, redness, inflammation, dry patches, or pallor. One treatment for certain sensitive skin conditions is hydrocortisone, a hormone. While it has excellent efficacy in treating skin sensitivities such as redness and inflammation, some users find it undesirable because it is not a so-called natural ingredient. Many of today's consumers desire to use skin treatment products that will ameliorate the adverse effects of sensitive skin, but prefer that these products contain natural or organic ingredients that are manufactured in a way that is environmentally friendly.

Skin treatment products containing botanical active ingredients for use on sensitive skin are known. However, one common problem when using botanical ingredients to treat sensitive skin is that the skin soothing components of the botanical extract are present in minute amounts. Accordingly, these products may not be as effective in treating sensitive skin as products that contain cortisone or other synthetic anti-inflammatory ingredients.

Accordingly, cosmetics companies are continually looking for new and improved skin treatment products for treating sensitive skin containing active ingredients derived from naturally occurring botanical ingredients which exhibit the same efficacy as products formulated with synthetic ingredients.

It has been discovered that compositions formulated with the combination of naturally occurring extracts from Hippophae and Pleurotus genuses provide excellent skin treatment products for improving undesirable conditions found in sensitive skin.

Accordingly, it is an object of the invention to provide a skin treatment product comprising a combination of extracts from the Hippophae and Pleurotus genuses.

It is another object of the invention to provide a method for treating sensitive skin for improvement by topically applying a composition containing a combination of extracts from the Hippophae and Pleurotus genuses.

### SUMMARY OF THE INVENTION

The invention is directed to a skin treatment composition comprising a combination of at least one extract from the Hippophae genus and at least one extract from the Pleurotus genus.

The invention is further directed to a method for treating sensitive skin for improvement by administering a topical composition comprising a combination of at least one extract from the Hippophae genus and at least one extract from the Pleurotus genus.

The invention also comprises a method for treating sensitive skin for improvement comprising the steps of:

cleansing the skin

applying a skin treatment composition comprising at least one extract from the Hippophae genus and at least one extract from the Pleurotus genus; and

applying a skin moisturizer to the skin which does not contain at least one extract from the Hippophae genus and at least one extract from the Pleurotus genus.

### DETAILED DESCRIPTION

### I. Definitions

All percentages used herein are percentages by weight unless otherwise indicated.

The term “adhesion inhibitor” means a particular ingredient has activity in inhibiting the adhesion pathway, which is the pathway by which cells adhere to blood vessels and other skin tissues when injury or immune challenge has occurred.

The term “certified” when used in connection with the term “organic” means that the organic ingredient or product was certified in accordance with 7 C.F.R. §205.400 et seq., hereby incorporated by reference in its entirety, or Ecocert®, the European control and certification authority.

The term “chemotaxis inhibitor” means a particular ingredient has activity in inhibiting the chemotaxis pathway, that is the pathway where chemical signals cause inflammatory cells to migrate toward the site in the skin or tissue where immune challenge has occurred.

The term “improvement’ when referring to treating the skin means that after treatment the skin will exhibit an improved appearance with respect to properties such as texture, feel, hydration, smoothness, blemishes, clarity, reduction in redness or inflammation, etc.

The term “normal” or “normalized” with respect to skin means that it is in its optimal state of natural health.

The term “organic” when used herein means that the ingredient or material complies with 7 C.F.R. §205, hereby incorporated by reference in its entirety, with respect to the preparation, manufacture, disposal, etc.

The term “sensitive skin” means skin that does not remain normalized after exposure to environmental assaults such as poor diet, fatigue, stress, pollution, sunburn, windburn, cold or hot weather, insect bites, cuts, exposure to allergens in food, water, or air, and so on; which in turn may result in skin dryness, blemishes, uneven pigmentation, redness, inflammation, scaliness, pallor, or manifestations of aging such as increase in line and wrinkle formation.

### II. The Compositions

The compositions of the invention contain at least one extract from the genus Hippophae and at least one extract from the genus Pleurotus.

A. The Extract From the Genus Hippophae.

The Hippophae genus, also referred to as sea buckthorns, are deciduous shrubs that are indigenous to Europe and Asia and grow close to sea shores. Suitable extracts include those from Hippophae Rhamnoides or Hippophae Salicifolia. The extracts may be derived from roots, stems, leaves, berries, seeds, or other portions of the plant. The extract may be in the form of a powder, aqueous or aqueous/alcoholic extract, or an oil or wax. The extract from the Hippophae genus is preferably present in the composition ranging from about 0.00001 to 25%, preferably from about 0.0001 to 20%, more preferably from about 0.001 to 15% of the composition. One form is a powder extract that may be purchased from Draco Natural Products Inc. either neat or in the form of a blend with glutathione, and extracts from Curcuma Longa, Panax Ginseng, Centella Asiatica, and Andrographis Paniculata. Also suitable is an oil carbon dioxide extracted from the berries of Hippophae Rhamnoides which may be purchased from Flavex Naturextrakte GmbH, Nordstrasse 7, Berlin, Germany. This form is a clear oil that is liquid at room temperature (25° C.) and orange red in color having a density ranging from about 0.9 to 1.0 g/cm³. Other oil extracts may be purchased from Barnet Products which is a mixture of 40 parts of Hippophae Rhamnoides oil and 60 parts olive oil. Preferred is where the extract is organic, more preferably certified organic.

In one preferred embodiment the extract is either a chemotaxis inhibitor or a adhesion inhibitor or both.

Preferred is an extract from Hippophae Rhamnoides in the powder or oil form.

B. The Extract From the Genus Pleurotus.

The composition of the invention also contains at least one extract from the genus Pleurotus, which are gilled mushrooms, the most common of which is the oyster mushroom. A variety of Pleurotus species are suitable including Pleurotus Acerosus, Pleurotus Astralis, Pleurotus Citrinopileatus, Pleurotus Cornucopiae, Pleurotus Cystidiosus, Pleurotus Djamor, Pleurotus Dryinus, Pleurotus Eryngii, Pleurotus Euosmus, Pleurotus Ferulae, Pleurotus Gardneri, Pleurotus Nebrodensis, Pleurotus Ostreatus, Pleurotus Pulmonarius, Pleurotus Tuberregium. Particularly preferred is an extract from Pleurotus Ostreatus also referred to as the tree oyster mushroom. The extract may be in the form a powder which can be purchased from Draco Natural Products Inc. or Actives International (the latter having the common name oyster mushroom polysaccharide. Also suitable are liquid extracts from vendors such as Fungi Perfecti in Olympia, Wash., which is an extract comprising from about 93 to 99% water with the remainder solids obtained from oyster mushroom. Preferred is where the extract is organic or certified organic. The extract is present in the composition in amounts ranging from about 0.00001 to 25%, preferably from 0.0001 to 20%, more preferably from about 0.001 to 15%.

In one preferred embodiment the Pleurotus extract may be a chemotaxis inhibitor or an adhesion inhibitor.

Preferred is an extract from Pleurotus Ostreatus in the powder or liquid form.

### III. Other Ingredients

The composition of the invention may contain a variety of other ingredients including but not limited to oils, humectants, other botanical extracts, preservatives, thickening agents, fatty acids, and so on. The compositions may be liquid, semi-solid or solid form, in the form of serum, lotion, crème, powder, cake, stick, gel, aqueous solution or dispersion and the like.

1. Other Botanical Ingredients

The composition may contain one or more additional botanical ingredients including those in the liquid, powder, solid or semi-solid form. If present, they may be present in amounts ranging from about 0.00001 to 20%, preferably from about 0.0001 to 10%, preferably from about 0.0005 to 8%. The ingredients may be extracted from the leaves, seeds, berries, stems, roots, or branches of plants. Examples of suitable botanical ingredients include, but are not limited to those from the genuses Olea, Hypsizigus, Limnanthes, Mangifera, Ocimum, Silybum, Tritcum, Citrus, Selaginella, Inonotus, Rosmarinus, Oenothera, Helianthus, Curcuma, Pelargonium, Lavendula, Cordyceps, Zingiber, Ganoderma, Elaeis, Glycyrrhiza, Salix, Macrycycstis, Pyrus, Saxifraga, Vitis, Morus, Scutellaria, Anthemis, Salvia, Rosmarinus, Panax, Siegesbeckia, Fructus, Ascophyllum, Bifida, Soja, Beta, Haberlea, Polygonum, Vitis, Humulus, Punica, Asparagopsis, Menyanthes, Hordeum, Cucumis, Evernia, and the like.

More specifically, suitable botanical ingredients include Olea Europa (Olive) oil, Yeast extract, Hypsizigus Ulmarius extract, Limnanthes Alba (Meadowfoam) seed oil, Mangifera Indica (Mango) seed butter, Butyrospermum Parkii (shea) butter, Ocimum Sanctum leaf extract, Silybum Marianum fruit extract, wheat (Triticum Vulgare) bran extract, Olive (Olea Europaea) extract, Citrus Grandis (grapefruit) peel extract, Selaginella Tamariscina (Spike Moss) extract, Inonotus Obliquus (mushroom) extract, Rosmarinus Officinalis (Rosemary) leaf extract, Mangifera Indica (Mango) leaf extract, Oenothera Biennis (Evening Primrose) oil, Citrus Aurantium Dulcis (Orange) oil, Helianthus Annus (Sunflower Seed) oil, Curcuma Longa (Turmeric) root extract, Citrus Nobilis (Mandarin Orange) peel oil, Pelargonium Graveolens flower oil, Lavendula Augustifolia (Lavendar) oil, Cordyceps Sinensis extract, Zingiber Officinale (Ginger) root extract, Ganoderma Lucidum extract, Camella Sinensis (Green Tea), Elaeis Guinensis, Glycyrrhiza Glabra, Salix Nigra, Macrocycstis Pyrifera, Pyrus Malus, Saxifraga Sarmentosa, Vitis Vinifera, Morus Nigra, Scutellaria Baicalensis, Anthemis Nobilis, Salvia Sclarea, Rosmarinus Officianalis, Citrus Medica Limonum, Panax Ginseng, Siegesbeckia Orientalis, Fructus Mume, Ascophyllum Nodosum, Bifida Ferment lysate, Glycine Soja extract, Beta Vulgaris, Haberlea Rhodopensis, Polygonum Cuspidatum, Citrus Aurantium Dulcis, Vitis Vinifera, Selaginella Tamariscina, Humulus Lupulus, Citrus Reticulata Peel, Punica Granatum, Asparagopsis, Curcuma Longa, Menyanthes Trifoliata, Hordeum Vulgare, Cucumis Sativus, Evernia Prunastri, Evernia Furfuracea, and mixtures thereof.

Particularly preferred are additional extracts from mushrooms such as Hypsizigus Ulmarius, Inonotus Obliquus, Ganoderma Lucidum, Cordyceps Sinensis, and mixtures thereof either alone or in combination with one or more of the other extracts mentioned herein.

2. Oils

The composition may contain oils in the form of pourable liquids. Such oils may be synthetic or naturally occurring and may be organic. If present the oils may range from about 0.1 to 50%, preferably from 0.5 to 40%, more preferably from about 1 to 35% of the composition.

Suitable silicone oils may be volatile or non-volatile, and may have a viscosity ranging from about 0.5 to 100,000 centistokes at room temperature. Examples of volatile silicone oils include linear or cyclic volatile silicones having a viscosity ranging from about 0.5 to 5 centistokes at room temperature. Examples of linear volatile silicones include hexamethyldisiloxane, octamethyltrisiloxane, decamethyltetrasiloxane, dodecamethylpentasiloxane and the like. Cyclic volatile silicones that may be used are cyclopentasiloxane, cyclohexasiloxane, or mixtures thereof.

Examples of linear non-volatile silicones include dimethicone, phenyl trimethicone, diphenyl dimethicone, trimethylsiloxyphenyl dimethicone, cetyl dimethicone, amodimethicone, phenyl dimethicone, and so on.

Also suitable are organic oils which may be in the form of volatile paraffinic hydrocarbons such as isododecane, isohexadecane; or non-volatile hydrocarbons such as polybutene, polydecene, squalane, or hydrogenated forms thereof respectively.

Other types of organic oils include esters of C6-30 straight or branched chain, saturated or unsaturated fatty acids and C1-10 mono-, di-, or polyhydric alcohols. Also suitable are triglycerides of fatty acids such as capric, caprylic, myristic, linoleic, linolenic, stearic, or behenic acids.

Also suitable are cholesterol, cholesterol sulfate, and the like.

3. Humectants

The composition may also contain one or more humectants, if present, ranging from about 0.01 to 20%, preferably from about 0.05 to 15%, more preferably from about 0.1 to 10%. Examples of suitable humectants include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. Preferably, the humectants used in the composition of the invention are C₁₋₆, preferably C₂₋₄ alkylene glycols, most particularly butylene glycol or glycerin.

4. Thickeners

In many cases it is desirable to incorporate one or more thickening agents into the composition. If present, suggested ranges are from about 0.01 to 25%, preferably from 0.1 to 20%, more preferably from about 0.1 to 15%. These agents may form part of the water or oil phase if such phases are present in the composition. Suitable thickeners include silicone elastomers, polysaccharides, silicone gums or waxes, natural or synthetic organic waxes, montmorillonite minerals, silicas and silylates, and so on.

Examples of silicone elastomers include Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Coming's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crosspolymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK, each of which are herein incorporated by reference in its entirety.

Suitable polysaccharides include naturally derived materials such as agar, agarose, alicaligenes polysaccharides, algin, alginic acid, acacia gum, amylopectin, chitin, dextran, cassia gum, cellulose gum, gelatin, gellan gum, hyaluronic acid, hydroxyethyl cellulose, methyl cellulose, ethyl cellulose, pectin, sclerotium gum, xanthan gum, pectin, trehelose, gelatin, and so on.

Also suitable are certain types of acrylic polymers such as carbomer, C10-30 alkyl acrylates crosspolymer, or those sold under the trademarks Simulgel® or Aristoflex®, having CTFA names sodium acrylate/sodium acryloyldimethyltaurate copolymer or acrylamide/sodium acryloyldimethyltaurate copolymer.

Also suitable are silica, silica silylate, magnesium aluminum silicate, calcium carbonate, or montmorillonite minerals such as quaternium-18 hectorite or bentonite.

Preferred compositions are in the form of emulsions or aqueous solutions or dispersions. Aqueous solutions or dispersions will generally contain from about 1 to 99%, preferably from about 2-80%, more preferably from about 5 to 75% water and from about 0.00001 to 20% of the combination of extracts. In addition, the aqueous solution or dispersion may contain any one or more of the ingredients mentioned herein and in the amounts specified.

Emulsions may be in the form of water in oil or oil in water emulsions. Such emulsions generally contain from about 0.1 to 99%, preferably from about 0.5 to 95%, more preferably from about 1 to 90% water and from about 0.1 to 75%, preferably from 0.5 to 70%, more preferably from about 1 to 65% oil.

### IV. The Methods

The invention is directed to a method for treating sensitive skin for improvement comprising administering a topical composition comprising at least one extract from the Hippophae genus and at least one extract from the Pleurotus genus. The composition may be topically applied one or more times per day, preferably in the morning and in the evening prior to retiring. The composition is useful to treat sensitive skin conditions such as skin dryness, blemishes, uneven pigmentation, redness, inflammation, scaliness, pallor, or line and wrinkle formation

The composition may be applied after cleansing the skin with a suitable cleanser, either one having a cleansing surfactant, or an oil based cream cleanser. The composition of the invention is best applied to clean skin. Most desirably the user then applies a regular moisturizer and other beauty products such as primer, foundation makeup, and other color cosmetic products. Preferably the remaining beauty products applied after application of the invention composition do not contain the Hippophae and Pleurotus extracts.

The composition of the invention has excellent properties in improving skin conditions associated with sensitive skin.

The invention will be further described in connection with the following examples which are set forth for the purposes of illustration only.

### Example 1

A skin treatment serum was prepared as follows:

| Ingredients | % by weight |
|---|---|
| Water | QS100 |
| Simmondsia Chinensis (Jojoba) Seed Oil | 8.00 |
| Glycerin | 4.50 |
| Yeast extract | 4.00 |
| Caprylic/capric/myristic/stearic triglyceride | 3.00 |
| Olea Europaea (Olive) fruit oil | 3.00 |
| Hypsizigus Ulmarius extract | 2.50 |
| Limnanthes Alba (Meadowfoam) seed oil | 2.00 |
| Hydrogenated lecithin | 1.00 |
| Mangifera Indicia (Mango) seed butter | 1.00 |
| Phenoxyethanol/chlorophenesin/glycerin/sorbic acid | 1.00 |
| Butyrospermum parkii (shea butter) | 1.00 |
| Hydrogenated lecithin | 0.80 |
| Dimethicone | 0.70 |
| Xanthan gum | 0.50 |
| Butylene glycol/water/Ocimum Sanctum leaf | 0.50 |
| extract/Silybum Marianum fruit extract | |
| Magnesium aluminum silicate/silica/calcium carbonate | 0.25 |
| Linoleic acid | 0.20 |
| Wheat (Triticum Vulgare) bran extract/olive (Olea | 0.20 |
| Europaea extract) | |
| Citrus Grandis (grapefruit) peel extract | 0.20 |
| Cholesterol | 0.20 |
| Selaginella Tamariscina (spike moss) extract | 0.10 |
| Inonotus Obliquus (mushroom) extract/cellulose | 0.10 |
| Rosmarinum Officinalis (rosemary) leaf extract | 0.10 |
| Mangifera Indicia (Mango) leaf extract | 0.10 |
| Hippophae Rhamnoides extract | 0.10 |
| Oenothera Biennis (evening primrose) oil | 0.10 |
| Citrus Aurantium Dulcis (orange) oil | 0.06 |
| Pelargonium Graveolens flower oil | 0.023 |
| Lavendula Augustifolia (lavender) oil | 0.023 |
| Sodium hyaluronate | 0.02 |
| Phenoxyethanol | 0.018 |
| Cordyceps Sinensis extract | 0.01 |
| Pleurotus Ostreatus (mushroom) extract | 0.01 |
| Zingiber Officinale (ginger) root extract | 0.01 |
| Ganoderma Lucidum extract | 0.01 |
| Olibanum | 0.008 |
| Pogostemon Cablin (patchouli) oil | 0.008 |

The composition was prepared by combining the ingredients and mixing well to form an emulsion.

### Example 2

The composition was tested on 80 females ranging in age from 20 to 60 years who had a range of skin types and who were users of prestige, specialty or physician branded anti-aging serum more than five days a week. All of the panelists used department store brand skin care products and used at least one natural or organic skin, makeup, or body product. Fifty of the panelists stated that they believed their skin was sensitive or reactive, defined as experiencing conditions such as redness, flushed skin, or rosacea. The panelists were instructed to use the invention composition on their face in the morning and evening on clean skin in place of their usual facial serum, and to apply the composition followed by their regular skin care regimen. The panelists completed a questionnaire after one week of use and at the conclusion of the four week study period. The study results are summarized below:

| | % of Total | % of Sensitive |
|---|---|---|
| | Panelists | Skinned Panelists |
| Perceived Positive Effect | Reporting | Reporting |
| Improvement in skin condition | 71 | 76 |
| Improvement in skin appearance | 75 | 80 |
| Improvement in skin texture and | 80 | 84 |
| feel | | |
| Reduction in number of skin | | 64 |
| reactions or flare ups | | |
| Reduced severity of skin reactions | | 70 |
| or flare ups | | |
| Reduction in overall duration of | | 66 |
| skin reactions or flare ups | | |

The above data demonstrates that panelists reported improved skin condition, appearance, and texture, and a reduction in sensitive skin conditions such as skin reactions, flare ups, and the like.

### Example 3

The test for determining whether one or more of the Hippophae or Pleurotus extracts is a chemotaxis inhibitor is conducted as set forth herein.

One test that may be used to determine whether a particular ingredient inhibits the Chemotaxis Pathway is one that is based upon the ability of a test material to inhibit the migration of PMNs toward a known chemotactic agent. To perform this test, heparinized peripheral venous blood (20-30 ml) is collected from healthy human donors who are requested to refrain from caffeine intake for 12 hours prior to the blood drawing. The blood sample is layered over a density gradient (mono-poly resolving media, ICN Pharmaceuticals, Costa Mesa, Calif.) and spun at 400×g for 30 min. The PMN rich fraction is removed and the red blood cells lysed with hypotonic saline. The PMN are washed twice with Hank's balanced salt solution (HBSS) and then resuspended in 5.0 ml HBSS with ions supplemented with 0.4% bovine serum albumin (Sigma). The concentration of cells is adjusted to 10×10⁶ PMN/ml. Collected PMNs are greater than 95% pure and 98% viable as assessed by the Trypan Blue Exclusion Assay, which is performed using a Boyden chamber apparatus with blind well chambers fitted with 5 mm pore size filters (Millipore). The apparatus consists of two vertical chambers separated by a filter that contains pores of a size chosen such that the holes are large enough for the cells to actively crawl through them but not so large that the cells can physically fall through into the lower chamber. PMN are then pre-incubated with test compounds at the indicated concentrations. A 200 ml PMN cell suspension is layered on the top of the filter, and 100 ml of chemotactic factors are added to the lower compartment. The chemo-attractant used in the assay is 0.125 nM LTB4. Following incubation at 37° C. for 90 minutes under a humidified atmosphere with 5% CO₂, the filters are fixed with propanol and stained with haematoxylin and eosin. The PMN chemotactic response is determined by the distance to the leading front and the number of cells that migrated to the front. The distance to the leading front is determined at 400× magnification by the distance the majority of the cells migrated through the filter. The results are expressed as the average number of cells per high powered field at the leading migratory front (PMN/HPF).

### Example 4

The test for determining whether one or more of the Hippophae or Pleurotus extracts is an adhesion inhibitor is conducted as set forth herein. The Adhesion Pathway is based upon the analysis of the adhesion of polymorphonuclear cells (PMN) to human dermal microvasicular cells—one of the things that occurs when leukocytes migrate to a site of irritation or infection in tissue that has been subject to assault. The test is conducted by collecting heparinized peripheral venous blood (about 20-30 ml) from healthy human donors who are requested to refrain from caffeine intake for 12 hours prior to the blood drawing. The heparinized blood from each individual is layered over a density gradient (Mono-Poly Resolving Media, sold by ICN Pharmaceuticals, Costa Mesa, Calif.) and spun at 400×g for 30 minutes. The PMN cell rich fraction is removed and the red blood cells lysed with hypotonic saline. The PMN fraction is washed twice with Hank's balanced salt solution (HBSS) and then re-suspended in 5.0 ml HBSS (with ions), which has been supplemented with 0.4% bovine serum albumin (Sigma Aldrich). The concentration of cells is adjusted to 10×10⁶ PMN/ml. Collected PMN are greater than 95% pure and 98% viable as assessed by the Trypan Blue Exclusion Test of Cell Viability, a test well known in the art. Human dermal microvasicular endothelial cells (HDMEC) are obtained from the Clonetics Corporation and maintained according to specifications until confluent. The PMNs at a concentration of 4×10⁶/ml are mixed 1:1 with the test material (final concentration of cells=2×10⁶/ml) and incubated for 30 minutes with the test material. The appropriate concentration of material to be tested is determined by conducting standard cytotoxicity tests to identify the highest concentration of test material that causes cytotoxicity. After this upper limit has been established successive serial dilutions of the test material are tested.

After a 30 minute incubation with the test material plus tetradecanoyl phorbol acetate (TPA, 5 ng/ml) or, for control, with test material alone, or with TPA alone or vehicle; the PMN (350,000/well) are added to wells of a 96-well microtiter plate in which endothelial cells are seeded at 20,000 cells/well and allowed to reach confluence. The endothelial cells are activated by pre-incubating with Interleukin-1β (10 U/ml) for 60 minutes at 37° C. in 5% CO₂. After the two cell types have been in contact for two hours the supernatant is removed, remaining cells gently rinsed, and 100 ml of 0.25% rose bengal (ICN) stain in PBS is added for 5 minutes at room temperature. Non-adherent cells are removed by two subsequent washes (Medium 199 with 25 mM HEPES and 10% fetal bovine serum). Stain incorporated into cells is released by the addition of 200 ml of a 1:1 solution of ethanol and PBS. After 30-45 minutes the wells are read in an ELISA reader (Bio-Tek Instruments Inc. Winooski, Vt.) at 570 nm. The level of adherence is given as the mean optical density reading at an OD570 for wells containing endothelial cells plus PMN minus the mean OD570 of wells containing endothelial cells alone.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

What we claimed is:

1. A skin treatment composition comprising a combination of at least one extract from the Hippophae genus and at least one extract from the Pleurotus genus.

2. The composition of claim 1 further comprising at least one additional extract from the genus Hypsizigus.

3. The composition of claim 2 further comprising at one additional extract from the genus Inonotus.

4. The composition of claim 3 further comprising at least one additional extract from the genus Ganoderma.

5. The composition of claim 1 wherein one or more of the extracts are chemotaxis or adhesion inhibitors.

6. The composition of claim 1 further comprising yeast extract.

7. The composition of claim 1 which is an aqueous based skin treatment composition comprising at least one extract from the genus Hippophae, at least one extract from the genus Pleurotus, at least one extract from the genus Inonotus, and at least one extract from the genus Ganoderma.

8. The composition of claim 8 comprising the extracts Hippophae Rhamnoides, Pleurotus Ostreatus, Inonotus Obliquus, and Ganoderma Lucidum.

9. The composition of claim 8 further comprising yeast extract.

10. The composition of claim 9 further comprising at least one fatty acid.

11. The composition of claim 10 further comprising at least one fatty acid triglyceride.

12. The composition of claim 11 further comprising dimethicone.

13. A method for treating sensitive skin for improvement comprising administering a topical composition comprising at least one extract from the Hippophae genus and at least one extract from the Pleurotus genus.

14. The method of claim 13 wherein one or more of the extracts are chemotaxis or adhesion inhibitors.

15. The method of claim 13 wherein the composition is applied twice per day.

16. The method of claim 13 wherein the composition is in the form of a liquid or semi-solid.

17. The method of claim 16 wherein the composition is in the form of a serum, lotion, crème, gel, stick, cake, or spray.

18. The method of claim 13 wherein the composition is applied prior to application of skin moisturizer or makeup.

19. The composition of claim 13 wherein the sensitive skin conditions treated are skin dryness, blemishes, uneven pigmentation, redness, inflammation, scaliness, pallor, or line and wrinkle formation.

20. A method for treating sensitive skin for improvement comprising the steps of:
cleansing the skin
applying a skin treatment composition comprising at least one extract from the Hippophae genus and at least one extract from the Pleurotus genus; and
applying a skin moisturizer to the skin which does not contain at least one extract from the Hippophae genus and at least one extract from the Pleurotus genus.
